(19) United States
(12) Patent Application Publication
(10) Pub. No.: US 2009/0182303 A1
(43) Pub. Date: Jul. 16, 2009

(54) Targeted Therapeutic Agent Release for Weight Loss Therapy

(76) Inventors: Steven Walak, Natick, MA (US); Michael Madden, Princeton, MA (US); Paul Smith, Smithfield, RI (US)

Correspondence Address: FAY KAPLUN & MARCIN, LLP, 150 BROADWAY, SUITE 702, NEW YORK, NY, 10038, US

(21) Appl. No.: 12/337,726

(22) Filed: Dec. 18, 2008

Related U.S. Application Data
(60) Provisional application No. 61/020,898, filed on Jan. 14, 2008.

(51) Int. Cl. A61K 9/22; A61K 38/00; A61M 25/10; A61K 9/00

(52) U.S. Cl. 604/500; 514/2; 424/423; 604/892.1; 604/103.01

(57) ABSTRACT

A method for treating obesity comprises anchoring at a first target site within a body a first therapeutic agent delivery device including a first therapeutic agent reservoir coupled to a first outlet which, when the first device is anchored at the first target site, is positioned adjacent to a first target treatment location within a GI tract of a patient and releasing a first therapeutic agent from the first reservoir via the first outlet.

### PRIORITY CLAIM

This application claims the priority to the U.S. Provisional Application Ser. No. 61/020,898, entitled “TARGETED THERAPEUTIC AGENT RELEASE FOR WEIGHT LOSS THERAPY” filed Jan. 14, 2008. The specification of the above-identified application is incorporated herewith by reference.

### BACKGROUND

Obesity is a potentially life threatening condition afflicting ever increasing numbers of patients. The treatment of obesity often involves one or more of lifestyle changes, drugs and surgery. The drugs currently in use may be broadly classified as either appetite suppressants such as Sibutramine or Rimonabant, which decrease hunger and increase satiety or lipase inhibitors such as Orlistat, which impede the digestion of fats.

Anti-obesity drugs are conventionally taken orally, exposing all of the organs to the drug and thereby increasing the incidence of side effects which may be severe. For example, side effects associated with conventionally delivered drugs include heart palpitations, tachycardia, increased blood pressure, insomnia, diarrhea and nausea. In addition, a large portion of each dose of these drugs is wasted as the delivery is not directed to the digestive or nervous system target.

### SUMMARY OF THE INVENTION

In one aspect the invention is directed to a method for treating obesity, comprising anchoring at a first target site within a body a first therapeutic agent delivery device including a first therapeutic agent reservoir coupled to a first outlet which, when the first device is anchored at the first target site, is positioned adjacent to a first target treatment location within a GI tract of a patient and releasing a first therapeutic agent from the first reservoir via the first outlet.

### BRIEF DESCRIPTION OF DRAWINGS

FIG. 1 is a diagram representing a portion of a digestive system including a therapeutic agent delivery device according to an embodiment of the invention;

FIG. 2 is a diagram showing an embodiment of a therapeutic agent delivery device according to the invention;

FIG. 3 is a diagram showing an alternate embodiment of a therapeutic agent delivery device according to the invention;

FIG. 4 is a diagram showing an alternate embodiment of a therapeutic agent delivery device according to the invention;

FIG. 5 is a diagram representing a portion of a digestive system comprising a stent according to an embodiment of the invention;

FIG. 6 is a diagram representing a portion of a digestive system with a therapeutic agent pump according to an embodiment of the invention;

FIG. 7A is a diagram representing a stomach and an embodiment of a therapeutic agent delivery device according to the invention;

FIG. 7B is a diagram representing a stomach and a further embodiment of a therapeutic agent delivery device according to the invention;

FIG. 8 is a diagram representing a duodenum and an embodiment of a therapeutic agent delivery device according to the invention;

FIG. 9 is a diagram representing an alternate embodiment of a therapeutic agent delivery device according to the invention;

FIG. 10 is a diagram representing yet another alternate embodiment of a therapeutic agent delivery device according to the invention;

FIG. 11 is a diagram representing an alternate embodiment of a therapeutic agent delivery device according to the invention;

FIG. 12 is a diagram representing a stomach and yet another embodiment of a therapeutic agent delivery device according to the invention;

FIG. 13 is a diagram representing yet another an alternate embodiment of a therapeutic agent delivery device according to the invention;

FIG. 14A is a diagram representing yet another an alternate embodiment of a therapeutic agent delivery device according to the invention;

FIG. 14B is a diagram representing an attachment means for the therapeutic agent delivery device according to the invention;

FIG. 14C is a second diagram representing an attachment means for the therapeutic agent delivery device according to the invention;

FIG. 15 is a diagram representing yet another an alternate embodiment of a therapeutic agent delivery device according to the invention; and

FIG. 16 is a diagram representing yet another an alternate embodiment of a therapeutic agent delivery device according to the invention.

### DETAILED DESCRIPTION

The present invention may be further understood with reference to the following description and to the appended drawings, wherein like elements are referred to with the same reference numerals. The present invention relates to devices and methods for targeted delivery of therapeutic agents such as drugs, nutritional products, etc. to the digestive system. In particular, the invention relates to delivering therapeutic agents to the digestive system to treat obesity.

The present invention provides methods and devices to improve the effectiveness of therapeutic agents and minimize their side effects by delivering controlled dosages directly to target sites in the body. For example, for the delivery of therapeutic agents for the treatment of obesity, a therapeutic agent release device is implanted in or near the digestive organs. The therapeutic agent release device comprises, for example, a therapeutic agent reservoir and a pump to dispense a therapeutic agent, wherein an outlet of the therapeutic agent reservoir is placed adjacent to or in contact with the target tissue. The therapeutic agent release device may be implanted using conventional surgical techniques or alternatively, may be implanted using minimally invasive endoscopic or laparoscopic methods, as those skilled in the art will understand. In one exemplary embodiment, the therapeutic agent release device may be implanted in the stomach or the small intestine such that an outlet of the reservoir communicates directly with the GI tract or the common bile duct (CBD) for release of controlled dosages of a therapeutic agent thereto.

A therapeutic agent release device according to the present invention may comprise a reservoir holding a therapeutic agent and a pump for supplying the therapeutic agent from the reservoir to a target site in the GI tract. As will be described in greater detail below, a single pump may be coupled to a plurality of reservoirs (e.g., via a valving arrangement) to supply separate dosages of different therapeutic agents at different times or to supply combinations of therapeutic agents, etc. Alternatively, a plurality of pumps may be coupled to a common reservoir so that each pump may supply the same therapeutic agent to different target sites as desired. Still further, a single pump may include multiple outlet conduits and a valving arrangement controllable so that the single pump may supply one or more therapeutic agents or combinations thereof to a plurality of target sites as desired. Flow control from the reservoirs may be facilitated by precision orifice selection.

Alternatively, each pump of the therapeutic agent release device may be formed as a separate unit coupled to one or more separate reservoirs. Externally communicating ports may be placed through the wall of the stomach 100 to load and refill reservoir(s) with therapeutic agents. For example, a Percutaneous Endoscopic Gastrostomy (PEG) tube may be incorporated into the system according to the invention to allow loading of the therapeutic agent through the abdominal wall. In further embodiments, pumps and reservoirs may be surgically implanted at remote sites in the body with delivery lines connected to the a desired location in an organ. For example, the pumps and/or reservoirs may be implanted under the skin or muscle and may, in alternate embodiments, be located external to the body with delivery lines connected to a desired location in an organ.

The local therapeutic agent release device of the present invention may also enable the use of novel therapeutic compounds by reducing a required total systematic dose and eliminating negative side effects, as those skilled in the art will understand. Specifically, the local delivery of therapeutic agents may enable the use of experimental compounds such as melanocortin-4 receptor agonists, ghrelin, neuopeptide Y antagonists, melanin-concentrating hormone antagonists, peptide YY, and hydrophobic detergents of pH buffering compounds. As would be understood by those skilled in the art, the therapeutic agent may be selected to inhibit the function of the bile and pancreatic lipase to reduce fat digestion and caloric intake. Furthermore, the agent may serve to neutralize bile/ lipase, coat food to prevent the digestion thereof, temporarily coat the GI tract to prevent the digestion of food, slow the process of peristalsis by temporarily paralyzing the smooth muscle of the stomach and/ or cause a sphincter such as the Pyloric Sphincter and the Sphincter of Oddi, to remain shut or reduce outflow, as those skilled in the art will understand. It will be further understood by those of skill in the art that other therapeutic agents may be delivered by the device, for example appetite suppressants, etc. In different embodiments, the therapeutic agent delivered by the device may comprise a compound that affects neural signals of the digestive organs to control feelings of hunger and satiety. For example, the therapeutic agent may target the Vagus nerve or other bundles of nerves that carry signals between the GI tract and the brain.

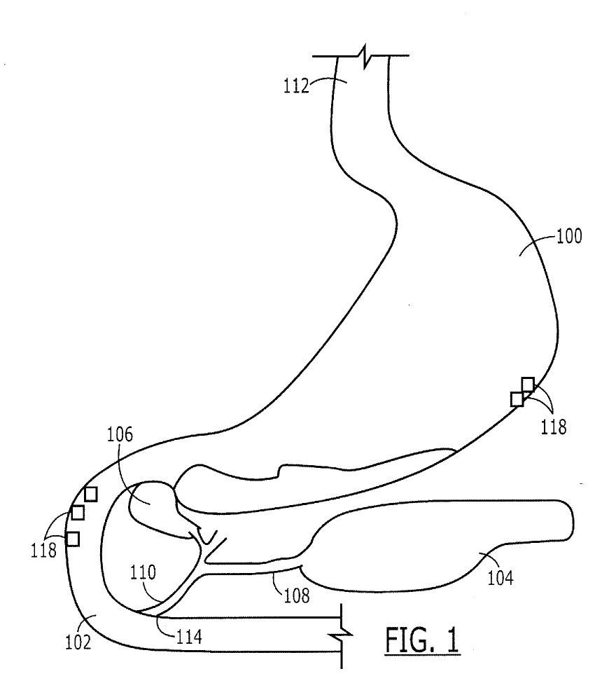

Embodiments of the present invention will be described with respect to the anatomy of the stomach 100 as referenced in FIG. 1, which shows a diagram of a portion of the digestive system with a therapeutic agent release device 118 according to the invention implanted within a stomach 100. Specifically, embodiments of the present invention may be implanted in the stomach 100 and within the duodenum 102, which is connected to the pancreas 104 via the pancreatic duct 108 and the major duodenal papilla 114. Additionally, the common biliary duct 110 connects the major duodenal papilla 114 to the gallbladder 106.

As those skilled in the art will understand, a material of the therapeutic agent release device 118 will be suitable to survive in the acidic environment of the stomach 100 and duodenum 102. Exemplary materials for the device include, but are not limited to: metals such as stainless steels, titanium, tantalum, cobalt alloys and nitinol and plastics such as Teflon, rubbers including neoprene, silicone, urethane, polyethylene, polypropylene, nylon, polycarbonate, polymethyl methacrylate, polyethylene terephthalate (PET) and polystyrene.

A positioning of the therapeutic agent release device 118 within the stomach 100 may correlate directly with the type of therapeutic agent being used. For example, therapeutic agents intended to impede the function of bile in the digestion of fat may be placed in the lower stomach, the pyloric antrum, the duodenal bulb or the common bile duct. Alternatively, agents targeting the vagus nerve to increase satiety may be placed in close proximity to highly innervated tissue or main nerve branches in the upper stomach or esophagus 112. The therapeutic agent release device 118 will be described in greater detail in reference to later embodiments, wherein any of the embodiments of the present invention may be implanted in the stomach 100 in the manner shown in FIG. 1.

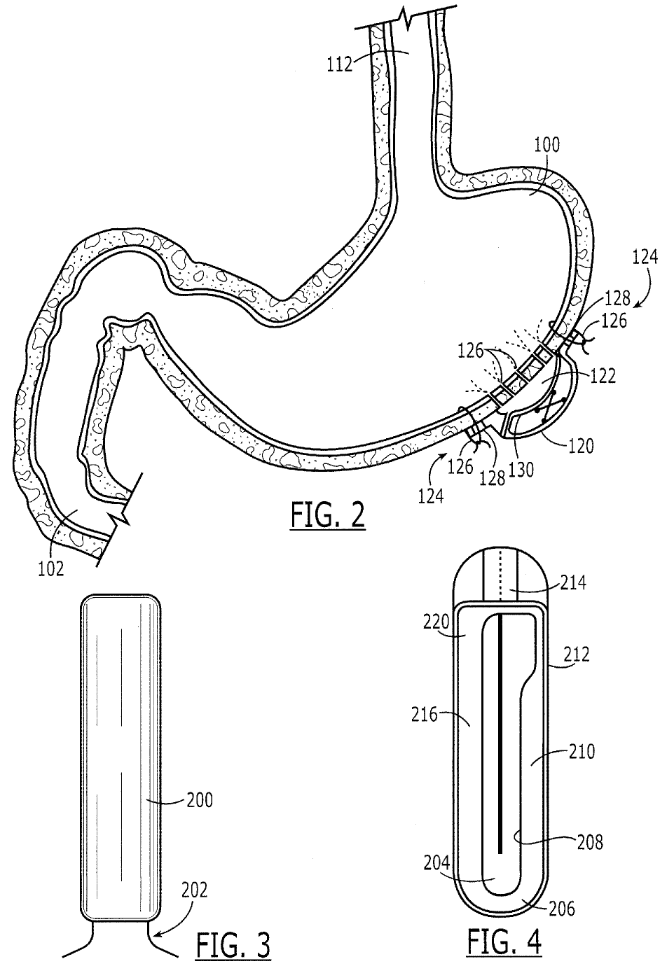

FIG. 2 shows one such exemplary embodiment of the present invention, wherein a therapeutic agent release device 120 is attached to an outer wall of the stomach 100. The therapeutic agent release device 120 comprises a reservoir 122 and a series of communicating conduits 126 which traverse through the wall of the stomach 100 to permit fluid communication between the stomach 100 and the reservoir 122 which contains an amount of a therapeutic agent The communicating conduits 126 may be microtube conduits that can further act as a restrictive means for metering the therapeutic agent. The therapeutic agent release device 120 may further be provided with a diaphragm 130 driven by one of internal springs and pressurized gas to meter out controlled doses of a therapeutic agent through a restricted orifice. One such pump is the pump manufactured by Infusaid Inc. This mechanically driven pumping system may include an internal power supply such as a battery to drive the mechanical pump while an external source of power recharges the battery (e.g., inductively) without necessitating a surgical procedure. Those of skill in the art will understand that other pumps and power supplies for pumping the therapeutic agent may be used according to the embodiments of the present invention. In an alternative embodiment, no communication conduits 126 may be employed and the therapeutic agents may be transferred across the tissue layers of the stomach 110.

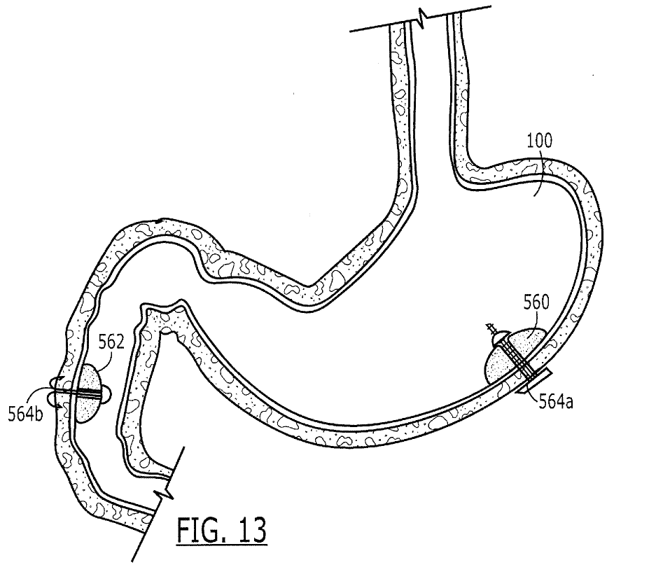

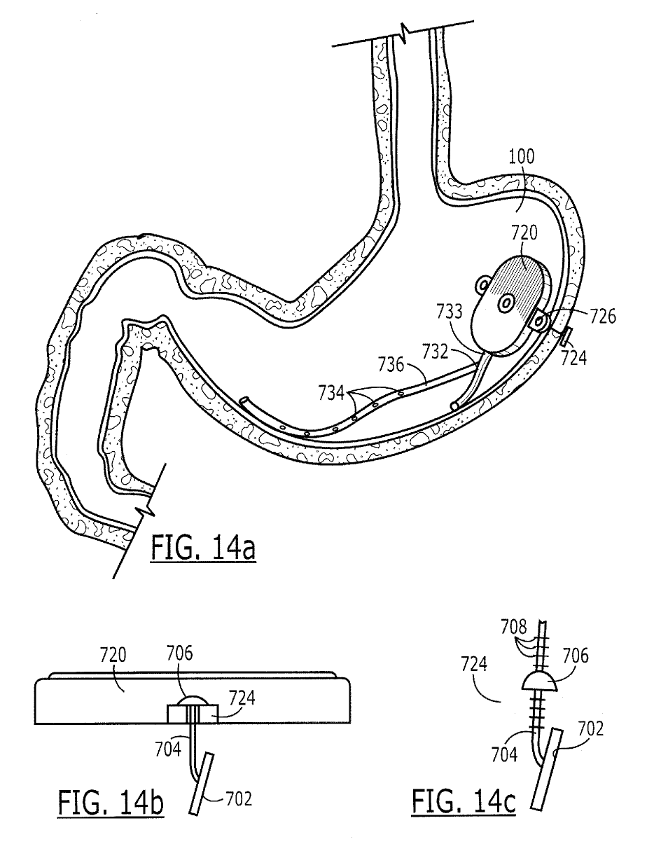

In operation, the therapeutic agent release device 120 may be traversed to a target portion of the stomach 100 and secured thereto via a securing means 124, wherein the securing means is one of a suture, hooks, screw, t-tacks or alternate means known in the art, as further detailed below with respect to FIGS. 13-14. In the embodiment shown, the securing means 124 comprises a suture 126 extending into the wall of the stomach. The suture 126 may be woven through and knotted over slots 128 formed on sides of the therapeutic agent release device 120. As described earlier, multiple therapeutic agent release devices 120 may be positioned at appropriate portion of the stomach 100.

As shows in FIGS. 3 and 4, a therapeutic agent release device 200 according to an alternate embodiment of the invention comprises one or more anchors 202 coupling the therapeutic agent release device 200 to tissue to retain a desired position. The anchors 202 may comprise retractable hooks, screws, or other mechanical devices, wherein mechanical features of the anchors 202 (e.g., curvatures, threading, etc.) may permit a secure attachment to tissue. As would be understood by those skilled in the art, the anchors 202 may be designed to degrade over a predetermined period of time, thus releasing the therapeutic agent release device 200 from the tissue attachment site. Once dislodged from the tissue, the therapeutic agent release device 200 may pass through and be excreted from the GI tract, thus obviating a need for a surgical removal of the device 200 after it has exceeded its useful life.

In an alternate other embodiment, a pump used with the therapeutic agent release device 200 may also degrade after a predetermined period of time has elapsed since implantation in the body

The therapeutic agent release device 200 may further comprise an osmotic pump 220 such as those manufactured by Alzet, Inc. The osmotic pump 220 comprises a fluid chamber 206 surrounded by an osmotic layer 210 forming a pumping chamber 216. A flexible impenneable membrane 208 containing a high concentration of a salt may be interposed between the fluid chamber 206 and the osmotic layer 210. Additionally, the osmotic layer 210 may be separated from the outside environment by a semipermeable membrane 212. A difference in solute concentration across the semipermeable membrane 212 draws water from the GI tract into the pumping chamber 216, thus expanding the osmotic layer 210 and applying pressure to the fluid chamber 206. The applied pressure forces a therapeutic agent 204 out through the outlet 214 at a controlled rate. For example, the rate of delivery of the therapeutic agent 204 may be directly related to and controllable by the permeability of the membrane 212 and/or the properties of the osmotic layer 210, as those skilled in the art will understand.

Employment of an osmotic pump is preferred since no external power supply is needed to release the therapeutic agent 204, which is pumped out of the therapeutic agent release device 200 by harnessing the properties of the materials of the osmotic pump 220 and the surrounding environment, as described in greater detail above. Additionally, substantially large quantities of the therapeutic agent 204 may be held in the fluid chamber 206 to provide long term therapy without the need to refill the fluid chamber 206, wherein the required volume of the fluid may vary depending on a dosage required for a particular patient.

In an alternate embodiment, the osmotic pump 220 may be composed of an electroactive polymer (“EAP”), shape memory material, etc. to facilitate a release of a therapeutic agent. Specifically, this osmotic pump 220 may be actuated via known means (e.g., application of a voltage thereto, etc,) to pump a therapeutic agent. In one embodiment, the osmotic pump 220 may be actuated to slowly pump a therapeutic agent to a target site over an extended period of time, wherein the rate of flow of the therapeutic agent may be increased or decreased by a factor at any time during the pumping process.

The osmotic pump 220 is sized for ease of insertion through an endoscope (not shown) and, preferably comprises a diameter smaller than an inner diameter of a working channel of the endoscope. As would be understood by those skilled in the art, the inner diameters of standard endoscope working channels are 2.0 mm., 2.8 mm., 3.7 mm., 4.2 mm. and 6.0 mm. The size of an osmotic pump 220 which does not need to be inserted through an endoscope working channel may be substantially larger than these values but is preferably small enough to facilitate insertion through the esophagus 112—i.e., the device should be smaller than a minimum diameter of the esophagus 112, which is typically about 25 mm. However, since endoscopes passed through the esophagus 112 are typically about 12 mm. to 14 mm. in diameter, osmotic pumps that comprise a larger size must be traversed to the target site via an alternate means. One such alternative is to traverse an endoscope to a first region within the body and manipulate a guidewire along the outside thereof to a target region, as those skilled in the art will understand. The endoscope may then be removed from the body and the osmotic pump 220 may be guided to the target region via the guide wire.

In an alternate embodiment, the osmotic pump 220 may be placed percutaneously or surgically at any of a plurality of sites on or in the body. In yet another alternate embodiment, the osmotic pump 220 may traverse through a trocar or a percutaneous endoscopic gastrostomy (“PEG”) tube, as those skilled in the art will understand, wherein the size of the osmotic pump 220 is smaller than the trocar or PEG tube. Furthermore, the osmotic pump 220 may be designed with a substantially small diameter relative to the size of a guiding mechanism being employed therewith, as noted above, but may comprise a longitudinal length that may increase as the diameter is reduced. Specifically, the osmotic pump 220 may assume a coiled or folded configuration during deployment and expand to a full length when positioned at a target treatment site.

Because of the small size and smooth shape of the therapeutic agent release device 200, the seed-like osmotic pump 220 of FIGS. 3-4 may be implanted within the GI tract using non-invasive techniques. Additionally, multiple therapeutic agent release devices 200 may be implanted at different locations on or in the body (e.g., different areas of the GI tract) wherein each of the multiple therapeutic agent release devices 200 may be loaded with either the same or different therapeutic agents as desired to apply different therapeutic agents or combinations thereof to the different treatment location. In an alternate embodiment, the therapeutic agent release device 120 may be formed with a substantially long and flexible body to allow for passage through an endoscope. Such a therapeutic agent release device may be passed through the endoscope and subsequently coiled along the wall of the GI tract to release the therapeutic agent over an extended period of time.

Different combinations of pumps and therapeutic agent reservoirs may be used to dispense therapeutic compounds according to the present invention. For example, in one embodiment the therapeutic agent may be released through the biodegradation of a polymer in which it is embedded. In this embodiment the therapeutic agent and the polymer are admixed to form a structure that degrades substantially uniformly to release the therapeutic agent at a desired rate, wherein the rate of release may be controlled by selecting an appropriate polymer. The degradable polymer may be used as a coating that produces a burst release when the polymer coating is breached.

Alternatively, as would be understood by those skilled in the art, various layers of different polymers may be employed to alter the rate of release of the therapeutic agent over time. Exemplary biodegradable polymers and polymer blends may include polyglycolide (“PGA”), poly(caprolactone), poly(dioxanone), copolymers of glycolide with trimethylene carbonate (“TMC”) and polyethylene glycol (PEG), wherein selection of a proper polymer may change the degradation rate from days to months. For example, changing the ratio of Poly(lactide) to Poly(glycolide) in a copolymer blend is known to dramatically alter the biodegradation rate thereof.

In another embodiment, the osmotic pump 220 may be designed as a rail with a plurality of separate spaces, each comprising a therapeutic agent therein, wherein the spaces may be sheathed by one of the polymer materials noted above. Accordingly, as each layer of the polymer material degrades in the body, the therapeutic agent housed in an adjacent space of the osmotic pump may be released into the body. In yet another embodiment, multiple pumps 220 may be employed in the therapeutic agent release device 200, wherein the plurality of pumps 220 may connect to one another (e.g., via a snap attachment means, etc.), thereby enabling the employment of more than one pump 220 in a particular location to affect the dosage of the therapeutic agent into the stomach.

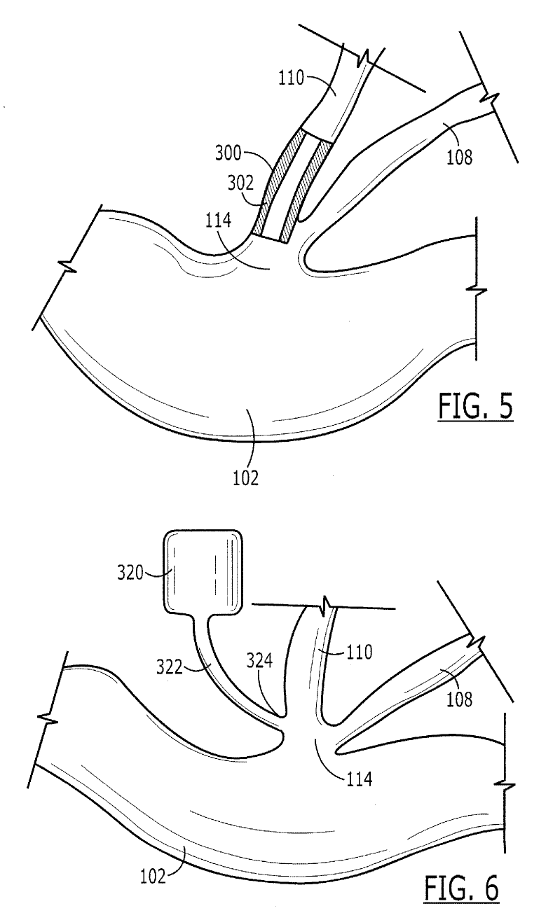

FIG. 5 depicts another embodiment of a therapeutic agent release device wherein a therapeutic agent such as a bile-neutralizing agent is delivered directly to the common biliary duct (“CBD”) 110. A therapeutic agent eluting stent 300 is implanted within the CBD 110 adjacent to an outlet to the duodenum 102. The stent 300 is formed as a substantially cylindrical member that substantially conforms to an outer wall of the CBD and is held therein using one of a frictional engagement and another securing means as described in greater detail with respect to FIGS. 2, 13 and 14. The stent 300 comprises a fluid reservoir 302 housing a therapeutic agent. The therapeutic agent can be delivered to the body by a pumping mechanism (not shown) open to an outlet port of the stent 300. The therapeutic agent eluting stent 300 releases a therapeutic agent that may include a lipase inhibitor, a bile neutralizer, detergent, pH buffering agent or other compound that reduces bile activity, impedes fat digestion and/or lowers the absorption of calories to a desired degree. In another embodiment, the therapeutic agent eluting stent 300 may release a detergent into the stomach to digest proteins, fats, or carbohydrates.

As shown in FIG. 6, a therapeutic agent pump and reservoir 320 according to a further embodiment of the invention is implanted near the CBD 110 and is connected thereto via a tube 322. In one embodiment, an end-to-side anastomosis 324 may be formed to connect the tube 322 to the CBD 110, thus providing a path for a therapeutic agent housed in the therapeutic agent pump and reservoir 320 to reach the GI tract. In one embodiment, the therapeutic agent pump and reservoir 320 is automatically controlled to deliver prescribed amounts of the therapeutic agent at preselected time intervals selected or determined based on data sensed by the system. For example, levels of gut hormones, actions by the patient or other external and/or biological stimuli may be sensed to trigger release of the therapeutic agent. Alternative embodiments may be designed to release therapeutic agents directly into the liver to facilitate metabolic activity.

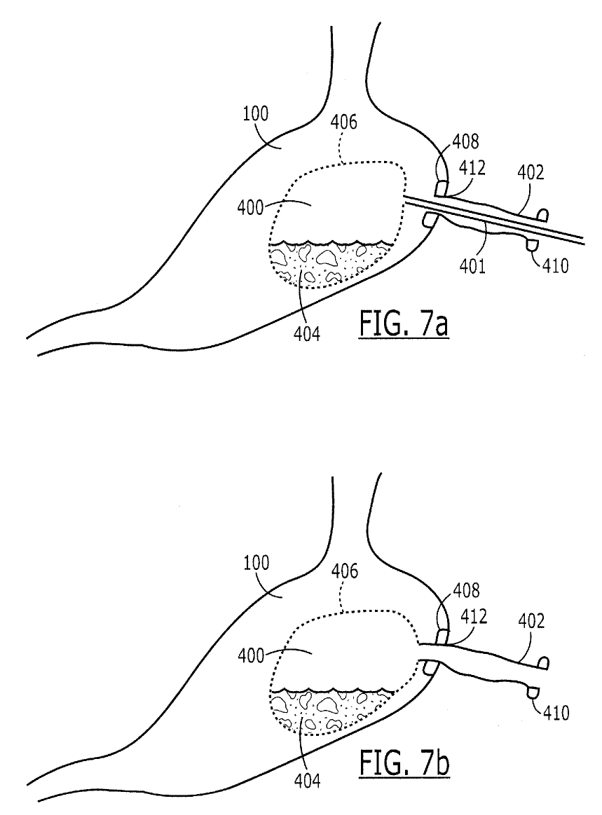

FIGS. 7A and 7B show two variations on an alternative system for treating obesity according to the present invention. As shown in FIG. 7A, a catheter 401 including a balloon 400 mounted at the distal end thereof is inserted into the stomach via a PEG tube 402. The PEG tube 402, which is placed into the stomach 100 using known techniques, includes a distal seal 408. After insertion into the stomach 100, the seal 408 is inflated and the PEG tube 402 is drawn proximally to seat the seal 408 against an inner wall of the stomach 100 to seal a perimeter of a stomach wall opening 412. A similar seal 410 formed at a proximal end of the PEG tube 402 is also inflated and advanced distally over the PEG tube 402 until seated around an outer wall of the stomach opening 412 into which the PEG tube 402 has been inserted. Alternatively, an external flange (not shown) situated on the PEG tube 402 may be used in place of the inflatable balloon seal 410.

Once the PEG tube 402 is fluidly sealed with the stomach 100, the catheter 401 is advanced through the PEG 402 and into the stomach 100 with the balloon 400 in a deflated state. An inflation fluid 404 is supplied to the balloon 400 via the catheter 401 to inflate the balloon 400 to occupy a desired volume within the stomach 100. The inflated balloon 400 decreases the volume of food required to cause satiety, thus reducing caloric intake. In addition, if desired, the balloon 400 may be formed of a material having a desired porosity so that the inflation fluid (e.g., a therapeutic agent) leaches from the balloon 400 into the stomach 100 at a desired rate. Exemplary materials that may be rendered porous may include, but are not limited to, silicone, ePTFE, polyisobutylene (SIBS), PeBAX, PET or urethane. In this case, the inflation fluid 404 is preferably a therapeutic agent acting on digestive fluids or structures of the digestive system to reduce caloric intake. Any of the therapeutic agents described above for application in the stomach may be used. For example, a therapeutic agent 404 utilized as the inflation fluid may include a drug or ghrelin antagonist.

In an alternate embodiment, only a portion 406 of a surface of the balloon 400 may be formed to be semipermeable so that the therapeutic agent is eluted therethrough to the stomach 100. Specifically, the semipermeable portion 406 of the balloon 400 may be formed of an otherwise impermeable material treated (e.g., using laser drilling or mechanical perforation) to form small pores therein. The semi-permeable material may be produced by leaching a constituent from a multi-constituent bulk material, phase inversion processing, foam processing or other known method for forming porous materials. As would be understood by those skilled in the art, the concentration of the therapeutic agent 404 and the volume of the balloon 400 may be tailored to the needs of individual patients.

As shown in FIG. 7B, in an alternate embodiment of the present invention, the balloon 400 may be mounted directly to the distal end of the PEG tube 402 instead of being selectively advanced to a target site via the catheter 401. In this embodiment, the balloon 400 may be selectively inflated with the inflation fluid 404 by introducing a fluid to the PEG tube 402 (e.g., via a catheter). Specifically, the PEG tube 402 of FIG. 7B projects distally beyond the distal seal 408 by a predetermined distance. Accordingly, when the inflation fluid 404 is supplied to the PEG tube 402, the balloon 400 inflates distally of the distal seal 408 and within the stomach 100.

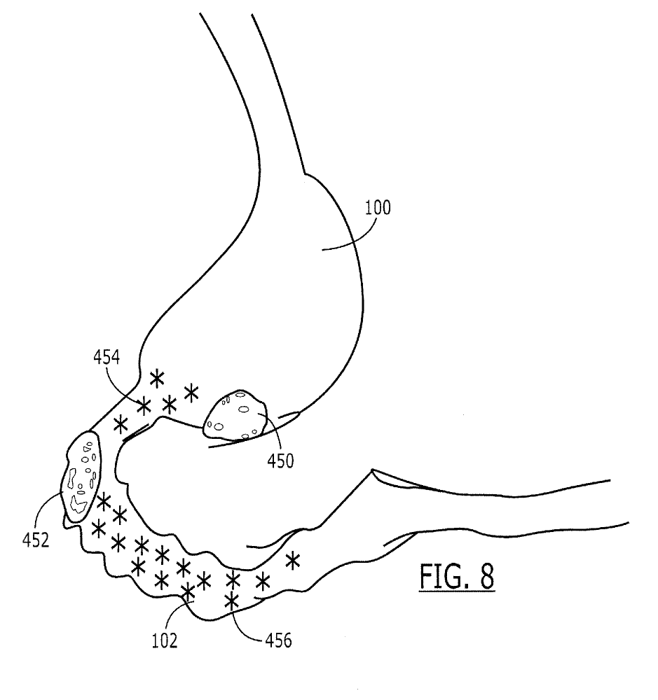

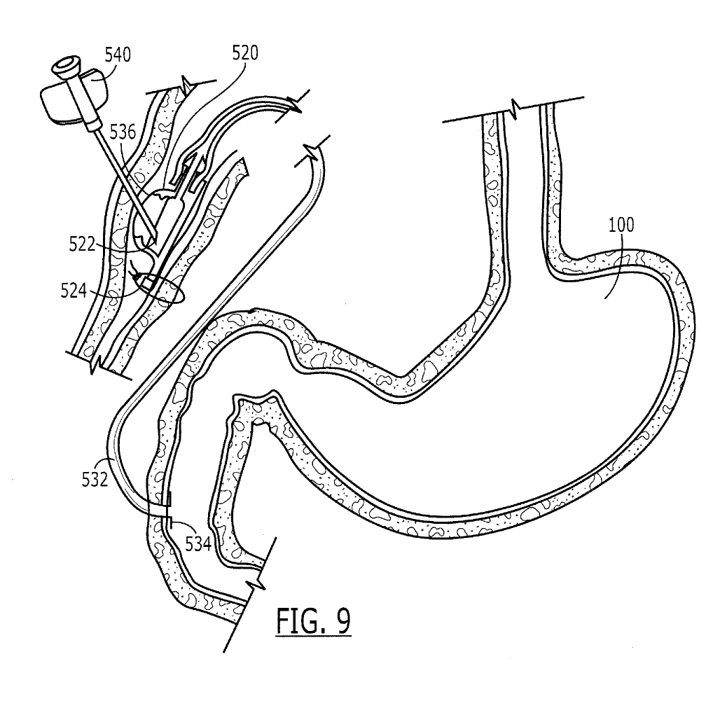

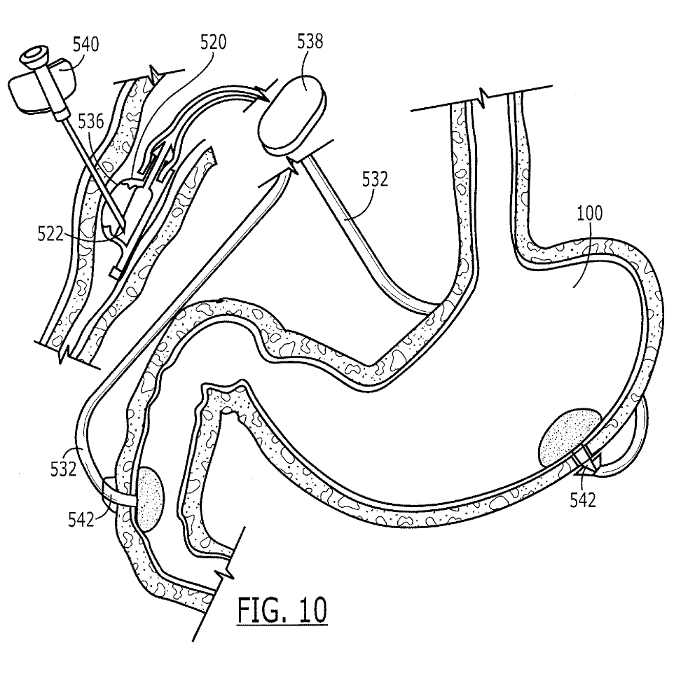

In another embodiment of the present invention, as shown in FIG. 8, the therapeutic agent release device may be formed as a tablet, balloon or other structure containing a therapeutic agent and releasing it at a controlled rate. Specifically, a first tablet 450 may be inserted into and implanted onto a wall of the stomach 100 and an optional second tablet 452 may be inserted into and implanted onto a wall of the duodenum 102 to release a therapeutic agent 454, 456 to inactivate certain digestion assisting enzymes. The first tablet 450 may be positioned in the stomach 100 adjacent to the pyloric sphincter so that the released therapeutic agent 454 passes quickly into the duodenum 102. The second tablet 452 may release the therapeutic agent 456 directly into the duodenum 102. The tablets 450, 452 comprise, for example, a porous or biodegradable outer portion housing the therapeutic agents 454, 456. The tablets 450, 452 may be anchored in the organ wall using degradable sutures, hooks, barbs, or screws. The attachment portions may be fabricated of one of a biodegradable polymer and a metal, wherein the biodegradable polymer may be selected from those previously described and the metal may include magnesium, iron, alloy steel, stainless steel or others which corrode in acidic environments. Alternatively, the first and second tablets 450, 452 may be inserted within the organ wall and held between tissue layers, as described in greater detail with respect to FIG. 11. Degradation rates of the tablets 450,452 and their respective attachment portions may be controlled through the application of coatings that are porous or which degrade slowly, as described in greater detail earlier.

FIG. 9 depicts an embodiment of the present invention wherein a therapeutic agent release device 520 is subcutaneously implanted near the outer surface of the skin. Specifically, the implantation site is selected for convenience and comfort of the patient as well as to facilitate access thereto for service, etc. such as, for example, the arm, the leg, the neck, etc. The therapeutic agent release device 520 may be fluidly connected and sealed to a conduit/catheter 532 which extends through the body to a target location in the stomach 100, duodenum 102, etc. The distal end of the conduit/catheter 532 opens to the stomach 100 and held against a wall thereof by a retaining member 534. The therapeutic agent release device 520 may be secured to the body by a securing member 524 similar to the securing devices disclosed in previous embodiments. In an alternative embodiment, all components of the implanted therapeutic agent release device 520 may be treated (e.g., by inclusion of holes, texture, or coatings) to promote tissue ingrowth to further fix the position of the therapeutic agent release device 520 within the body.

Implanting the therapeutic agent release device 520 in proximity to the surface of the skin facilitates refilling the therapeutic agent release device 520 which may, for example, include a selectively permeable septum 536 on an outer surface thereof facing the surface of the skin. When the supply of a therapeutic agent in the therapeutic agent release device 520 has been exhausted, a needle 540 fluidly connected to a source of therapeutic delivery agent may be inserted through the septum 536 to refill the reservoir 522. A pump or other means (not shown) in the therapeutic agent release device 520 may regulate the flow of the therapeutic delivery agent through the conduit/catheter 532 to the stomach 100 as would be understood by those skilled in the art.

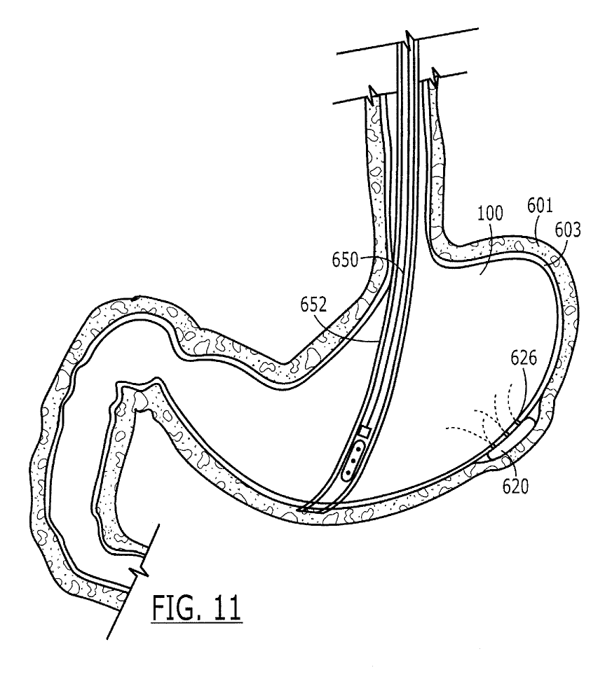

In an alternate embodiment, as shown in FIG. 10, the therapeutic agent release device 520 may comprise multiple conduits 532 extending to different target sites in the body. A distal end of the each of the conduits 532 may be provided with a retention member 542 which serves to retain the therapeutic agent in the conduit/catheter 532 until a predetermined fluid pressure is applied thereto by the pump 538. Furthermore, it is noted that, although this embodiment is shown with two conduits/catheters 532 extending from the pump 538 to target sites on the stomach, any number of conduits/catheters may be employed without deviating from the scope of the present invention.

The therapeutic agent release device 520 may further comprise a subcutaneous pump 538 situated along a length of one or both of the conduits 532 to regulate the flow of therapeutic agent therethrough to a target site or sites in the stomach. The pump 538 may be activated, for example, by a controller outside the body to release desired doses of therapeutic agent into the body using, for example, wireless transmission, induction, etc. Accordingly, the entire length of the conduit/catheter 532 may serve as a reservoir, increasing the available quantity of therapeutic agent which may be stored therein.

The aforementioned embodiments disclose a therapeutic agent release device that may be implanted either internally in the stomach 100, attached to an external wall thereof or implanted subcutaneously and fluidly connected thereto by one or more conduits. In another embodiment, a therapeutic agent release device 620 may be implanted within tissue layers of the wall of the stomach 100. Specifically, as shown in FIG. 11, the therapeutic agent release device 620 may be implanted between the serosa 601 and the mucosa 603 of the stomach 100 and may comprise a series of communicating conduits 626 to infuse a therapeutic agent into the stomach 100. Alternatively, the therapeutic agent release device 620 may comprise a therapeutic agent transferable across the mucosa 603 to enter the stomach.

The therapeutic release device 620 may be implanted via the use of an injection needle 652, an elongated catheter or other means as would be understood by those skilled in the art wherein a distal tip of the injection needle is inserted to a target location in the stomach, for example, via the esophagus 112. The injection needle 652 may be guided, for example, via the employment of an optical system or other guiding system located on a distal end of an endoscope. Once a target location has been reached and the distal tip of the injection needle 652 has been inserted to a target location between the serosa 601 and the mucosa 603, the therapeutic release device 620 is inserted through the device and propelled distally by an injection device 650. After the therapeutic release device 620 has been positioned at the target location, the injection needle 652 and endoscope (not shown) may be withdrawn from the esophagus 112. It is noted that the above noted method may be used to implant the therapeutic release device 620 in any hollow organ or lumen and is not limited solely to the injection of a device into the wall of the stomach.

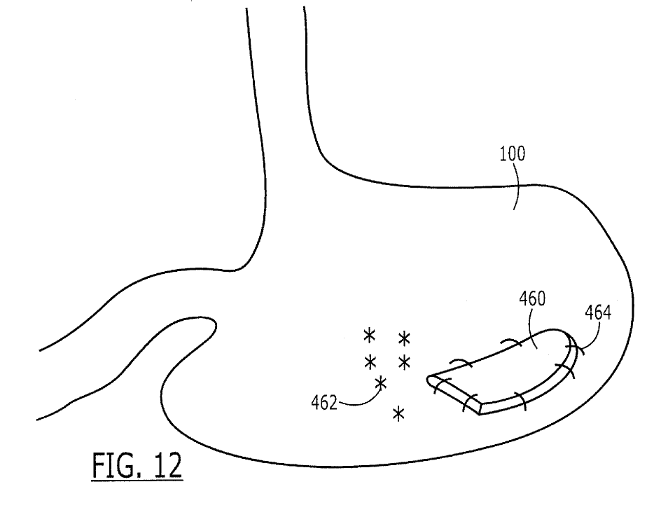

In another alternate embodiment, as shown in FIG. 12, an implant 460 formed as a tablet including an acid neutralizer 462 may be placed within the stomach 100, preferably further upstream of the pyloric sphincter, to reduce the degree to which food is broken down in the stomach 100 impeding absorption of calories. As would be understood by those skilled in the art, the implant 460 is preferably designed to provide a rate of release of the acid neutralizer 462 optimized to achieve a desired, substantially constant, long term reduction in food breakdown in the stomach 100. For example, porous and/or degradable portions of the implant 460 are preferably designed to release the therapeutic agent 462 at a controlled rate while hooks 464 or other mechanical means retain the implant 460 at a desired location within the GI tract. As noted above with respect to earlier embodiments, the hooks 464 may be formed of a degradable material so that the implant 460 is released from the target site after a selected amount of time has elapsed. The release rate of the implant 460 may vary depending on the specific requirements for a patient and procedure and may comprise one of a release rate of 0-3 months, 4-6 months or 6-9 months, wherein the initiation of the release may comprise a time delay so as to begin the release at a predetermined start time. For example, the implant 460 may be remotely triggerable by one of a directly connected switch and a remote activation system. For example, high-frequency ultrasound may be aimed at the implant 460 so that the elicited ultrasound waves initiate dissolution of the implant 460. The release rate and release initiation of the implant 460 may then determine how often the tablets are to be replaced.

As shown in FIG. 13, a porous implant 560 is formed to dispense metered therapy with different styles of the implant 560 being suitable for different target sites. The implant 560 of this particular embodiment are shown with a t-tack attachment means 564a, wherein the implant 564 is held in place against the stomach wall via a hooked attachment members 564b extending to the outside of the stomach wall, as described in greater detail with respect to FIGS. 14a-14c.

In yet another embodiment of the present invention, the therapeutic agent release device may deliver a therapeutic agent to multiple target sites simultaneously. As shown in FIG. 14a, a therapeutic agent release device 720 may be implanted in the stomach 100 via a t-tack member 724 described in greater detail below with respect to FIGS. 14b-14c. Specifically, the therapeutic agent release device 720 is positioned by the t-tack members 724 attached to slots 726 formed on lateral sides thereof. An outlet 732 may extend out of the therapeutic agent release device 720 to infuse a therapeutic agent into the stomach 100 at a predetermined perfusion rate. The outlet 732 may be formed as a tubular extension extending out of the therapeutic agent release device 720 by a predetermined distance. The outlet 732 may further comprise a port 733 to receive a conduit 736 to aid in the perfusion of the therapeutic agent through the stomach 100. Specifically, the conduit 736 may be formed as an elongated tubular element formed of a predetermined length suitable to reside within a stomach 100 of a patient. The conduit 736 may comprises substantially elastic properties to permit a substantial flexion thereof to conform to the anatomy of the stomach 100 without causing trauma thereto. The conduit 736 may thus extend adjacent a wall of the stomach by the predetermined distance, being held in position by_. The conduit 736 may further comprise a plurality of openings 734 disposed along a length thereof to aid in the distribution of the therapeutic agent to the stomach 100. The openings 734 may be sized and shaped to allow the therapeutic agent to diffuse therefrom at a predetermined diffusion rate. Thus, the therapeutic agent release device 720 permits the therapeutic agent to be evenly distributed through the stomach 100.

The t-tack member 724 comprises a substantially disc-shaped member 702 situated at a first end of a substantially flexible wire 704. The wire 704 comprises barbs 708 distributed along a length thereof, the barbs formed of a substantially flexible material exhibiting a predetermined rigidity. During insertion into the body (e.g., through an endoscope or catheter), the disc-shaped member 702 is bent to lie substantially parallel to a longitudinal axis of the wire 704, thus minimizing the profile of the t-tack member 724. Once a target site in the stomach is reached, the disc-shaped member 702 is positioned to lie substantially perpendicular to a longitudinal axis of the wire 704. The wire 704 is then pierced through a target portion of the wall of the stomach 100. The slot 726 of the therapeutic agent release device 720 is then threaded over the portion of the wire 704 received in the stomach. A locking cap 706 is then slidably placed over the wire 704. The locking cap 706 comprises an opening (not shown) sized and shaped to receive the wire 704 therethrough. The slidable insertion of the locking cap causes barbs 708 of the wire 704 to deflect to permit the locking cap 706 to slide therepast.

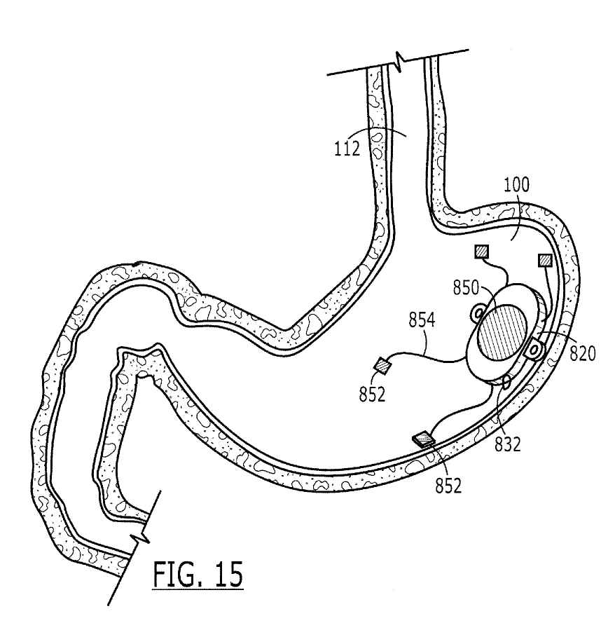

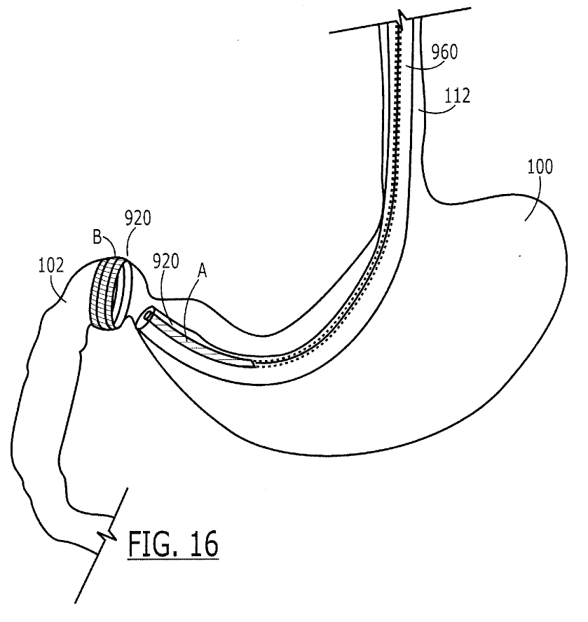

As shown in FIG. 15, a therapeutic agent release device 820 according to yet another embodiment of the invention comprises a mechanism for performing chemical sensing. Specifically, the therapeutic agent release device 820 is inserted to an appropriate site in the stomach 100 via the esophagus and attached to the wall of the stomach 100 via any of the previously disclosed methods including, for example, t-tack positioning, sutures, hooks, screws, etc. The main body of the therapeutic agent release device 820 may comprise, on its outer surface, a bulk food sensing means 850, wherein the bulk food sensing means 850 may be incorporated into a pump portion, wherein the pump portion would like in direct contact with the tissue of the stomach 100. Accordingly, the bulk food sensing means 850 senses the presence of food in the stomach 100 by measuring a tissue reaction to food entering the stomach (i.e., by monitoring movement of the esophagus 112). The therapeutic agent release device 820 may further comprise a plurality of leads 852 extending from the main body portion thereof and electrically connected thereto via a lead or wire 854. The leads 852 serve as a secondary food sensing mechanism extending to a plurality of sites along the wall of the stomach 100, thereby enhancing the accuracy in sensing tissue activity in response to food consumption, as those skilled in the art will understand.

The food sensing mechanism of the therapeutic agent release device 820 need not be connected to an external power supply. For example, they may be connected to an internal power supply such as a battery the life and size of which may vary depending on the length of time the therapeutic agent release device 820 is to remain in the body. A conduit 832 of the therapeutic agent release device 820 may serve as a restrictor for metering the therapeutic agent supplied to the stomach 100 and the device 820 may contain a refilling mechanism as described with respect to any of the earlier embodiments.

Once the bulk food sensing means 850 and/or the leads 852 detect a condition indicating that food has entered the stomach 100 (via the sensing of esophagus/tissue movement in the stomach 100), the pump of the therapeutic agent release device 820 may be activated, causing the reservoir to secrete a selected dosage of therapeutic agent into the stomach 100. The precise dosage may be controlled by the conduit 832, which serves as a restrictor.

In another embodiment of the present invention, a therapeutic agent release device 920 may during insertion into the stomach 100 via, for example, an endoscope 960, assume an elongated configuration A, facilitating the insertion of the therapeutic agent release device 920 through the esophagus 112. Once the therapeutic agent release device 920 has been inserted to a desired location, such as, for example, a location proximal to the duodenum 102, the therapeutic agent release device 920 may be deployed to a retention configuration B, in which the therapeutic agent release device 920 coils around a length of the duodenum 102. Accordingly, the therapeutic agent release device 920 may comprise a retractable coil design which automatically returns to the coiled retention configuration B upon the release of a force applied thereto to hold the therapeutic agent release device 920 in the elongated configuration A. The therapeutic agent release device 920 may be held in the elongated configuration A by any known means.

Those of skill in the art will understand that the therapeutic agent release device according to the present invention may be used to deliver different therapeutic agents to various areas of the patient's anatomy. In addition to delivering therapeutic agents to treat obesity to the stomach or duodenum, the embodiments of the invention may deliver therapeutic agents for the treatment of cancer and other diseases in the GI tract and other organs.

The present invention has been described with reference to specific exemplary embodiments. Those skilled in the art will understand that changes may be made in details, particularly in matters of shape, size, material and arrangement of parts. Accordingly, various modifications and changes may be made to the embodiments. The specifications and drawings are, therefore, to be regarded in an illustrative rather than a restrictive sense.

What is claimed is:

1. A method for treating obesity, comprising:
anchoring at a first target site within a body a first therapeutic agent delivery device including a first therapeutic agent reservoir coupled to a first outlet which is positioned adjacent to a first target treatment location within a GI tract of a patient when the first therapeutic agent delivery device is anchored at the first target site; and
releasing a first therapeutic agent from the first reservoir via the first outlet.

2. The method according to claim 1, wherein the first therapeutic agent delivery device includes a pump fluidly coupled between the reservoir and the first outlet to drive the first therapeutic agent out from the first reservoir via the first outlet.

3. The method according to claim 1, wherein the first target site is in one of a stomach and an intestine.

4. The method according to claim 1, wherein, when the first therapeutic agent delivery device is anchored at the first target site, the first outlet opens into a common bile duct.

5. The method according to claim 1, wherein the first target site is a nerve structure in the GI tract.

6. The method according to claim 5, wherein the first target site is a vagus nerve bundle.

7. The method according to claim 1, wherein the first therapeutic agent is one of a lipase inhibitor, a monoamine reuptake inhibitor, a CB₁ receptor blocker and a neutralizer of one of digestive acids and digestive enzymes

8. The method according to claim 2, wherein the pump is an osmotic pump.

9. The method according to claim 1, further comprising the step of implanting the first therapeutic agent delivery device through one of endoscopic, laparoscopic and open surgical procedures.

10. The method according to claim 2, further comprising implanting a second therapeutic agent delivery device at a second target site within the GI tract, the second therapeutic agent delivery device including a second therapeutic agent reservoir fluidly coupled to a second pump and a second outlet of the second pump which is positioned adjacent to a second target site within a GI tract of a patient when anchored at the second target site.

11. The method according to claim 10, wherein the second reservoir includes a second therapeutic agent.

12. The method according to claim 2, further comprising the step of recharging an implanted battery of the pump by inductive coupling to a power source which remains external to a body of the patient.

13. The method according to claim 1, further comprising implanting the therapeutic agent delivery device through a Percutaneous Endoscopic Gastrostomy (PEG) tube.

14. The method according to claim 1, wherein the first therapeutic agent is released by diffusion.

15. An implantable device, comprising:
a therapeutic agent reservoir sized and shaped for implantation within a living body;
a pump sized and shaped for implantation at a target site within the body, the pump being fluidly connected to the reservoir to motivate fluid from the reservoir to an outlet of the pump wherein, when implanted at a target site within the body, the outlet is located adjacent to target tissue to be treated by a therapeutic agent included within the reservoir; and
an anchoring element attaching the device to an anatomical structure at the target site in a desired position.

16. The device according to claim 15, wherein the target site is within one of a stomach and an intestine and wherein outer surfaces of the pump and the reservoir are formed of bio-compatible materials selected to resist corrosion due to contact with digestive fluids.

17. The device according to claim 15, wherein outer dimensions of the reservoir and the pump are selected to enable them to pass through a working channel of an endoscope.

18. The device according to claim 15, wherein the therapeutic agent is one of a lipase inhibitor, a monoamine reuptake inhibitor and a CB₁ receptor blocker.

19. The device according to claim 18, wherein the therapeutic agent is selected to alter the behavior of at least one nerve structure in a gastro-intestinal tract.

20. The device according to claim 15, wherein the pump is a mechanical pump and the implantable device further comprises an implantable battery powering the mechanical pump and an induction circuit coupled to the battery for transmitting to the battery power received inductively from an external power source.

21. The device according to claim 15, wherein the pump is an osmotic pump comprising an osmotic layer and a semipermeable membrane, properties of the osmotic layer and the semipermeable membrane being selected relative to properties of tissue adjacent to the target site to control a rate of release of the therapeutic agent.

22. The device according to claim 15, wherein the reservoir is refillable through an externally communicating port.

23. The device according to claim 15, wherein the anchoring element is formed of a material selected to degrade after a predetermined time has elapsed since implantation.

24. The device according to claim 15, wherein the therapeutic agent neutralizes one or both of digestion enzymes and digestive acids.

25. The device according to claim 15, wherein the therapeutic agent interferes with neural signals to digestive organs.

26. A system for treating obesity comprising:
a percutaneous endoscopic gastrostomy (PEG) tube; and
a balloon insertable into a stomach via the PEG tube, at least a portion of a surface of the balloon having a porosity selected to control a rate of elution into the stomach of an inflation fluid supplied to the balloon.

27. The system according to claim 26, further comprising a catheter including an inflation lumen extending therethrough to a distal opening, the balloon being coupled to a distal end of the catheter surrounding the distal opening.

28. A method for treating obesity comprising:
inserting into a GI tract an implant including a therapeutic agent, the therapeutic agent being embedded in one of a degradable portion and a porous portion of the implant for release therefrom at a selected rate; and
anchoring the implant at a target site.

29. The method according to claim 28, wherein the implant is a tablet anchored at the target site using a plurality of hooks formed of a material designed to degrade and release the tablet after a predetermined time has elapsed.

30. The method according to claim 28, wherein the implant is a therapeutic agent eluting stent and wherein the target site is within a common biliary duct, the therapeutic agent comprising one of a lipase inhibitor and a detergent.

31. The method according to claim 30, wherein the therapeutic agent comprises a bile neutralizer.
